(19) United States
(12) Patent Application Publication
(10) Pub. No.: US 2023/0099266 A1
(43) Pub. Date: Mar. 30, 2023

(54) RISK RELATIONSHIP RESOURCE ALLOCATION TOOLS

(71) Applicants: Christopher M. Horan, Simsbury, CT (US); Wendy S. Conklin, West Sacramento, CA (US); Jeffrey L. Laton, Gray, ME (US); Stephen J. Aloi, Glastonbury, CT (US); Satyanarayana Raju Datla, Irving, TX (US); Keren Shemesh, West Hartford, CT (US)

(72) Inventors: Christopher M. Horan, Simsbury, CT (US); Wendy S. Conklin, West Sacramento, CA (US); Jeffrey L. Laton, Gray, ME (US); Stephen J. Aloi, Glastonbury, CT (US); Satyanarayana Raju Datla, Irving, TX (US); Keren Shemesh, West Hartford, CT (US)

(21) Appl. No.: 17/490,341

(22) Filed: Sep. 30, 2021

(51) Int. Cl. G06Q 40/08; G06Q 10/10; G16H 70/20; G16H 40/20; G16H 10/60; G16H 50/20; G06Q 20/40; G06F 9/50; G06N 20/00

(52) U.S. Cl. CPC G06Q 40/08; G06Q 10/1057; G16H 70/20; G16H 40/20; G16H 10/60; G16H 50/20; G06Q 20/405; G06F 9/5083; G06N 20/00

(57) ABSTRACT

Some embodiments are associated with a system that provides an automated risk relationship resource allocation tool via a back-end application computer server of an enterprise. A resource allocation data store may contain electronic records representing requested resource allocations between the enterprise and a plurality of entities (collected from the entities and service providers). The server may then receive an indication of a selected requested resource allocation and retrieve, from the resource allocation data store, the electronic record associated with the selected requested resource allocation. The server may then automatically determine an initial duration for the selected requested resource allocation based on the shortest of: (i) an entity expected duration, (ii) a service provider expected duration, and (iii) a third-party guideline expected duration. Some embodiments may generate a final recommendation associated with an extension request for a requested resource allocation from an entity depending on whether service provider records are needed.

### BACKGROUND

It may be advantageous to analyze the risks and resource allocations associated with multiple systems and/or entities. For example, it might be advantageous to understand particular amounts of risk and allocations and the impact that such risks and allocations may have had on past (and, potentially, future) performance. Moreover, an enterprise might want to facilitate understanding and reaction to requests for allocations of resources—and a manual review of such requests may be an important part of this process. The breadth and depth of information associated with resource requests, often over an extended period of time, can overwhelm such a review process. That is, manually examining and understanding these types of risks and allocations associated with risk relationships can be a complicated, time consuming, and error-prone task, especially when there are a substantial number of inter-related systems, entities, and characteristics impacting resource allocations, and/or other factors involved in the analysis.

It would be desirable to provide systems and methods to provide an automated risk relationship resource allocation tool in a way that provides faster results in an improved way to ensure accuracy and consistency as compared to traditional approaches.

### SUMMARY OF THE INVENTION

According to some embodiments, systems, methods, apparatus, computer program code and means are provided to provide an automated risk relationship resource allocation tool in a way that provides faster results in an improved way to ensure accuracy and consistency as compared to traditional approaches and that allow for flexibility and effectiveness when responding to those results. The system may include a resource allocation data store that contains electronic records representing requested resource allocations between the enterprise and a plurality of entities (collected from the entities and service providers). The server may then receive an indication of a selected requested resource allocation and retrieve, from the resource allocation data store, the electronic record associated with the selected requested resource allocation. The server may then automatically determine an initial duration for the selected requested resource allocation based on the shortest of: (i) an entity expected duration, (ii) a service provider expected duration, and (iii) a third-party guideline expected duration. Some embodiments may generate a final recommendation associated with an extension request for a requested resource allocation from an entity depending on whether service provider records are needed.

Some embodiments comprise: means for receiving, by a back-end application computer server from a resource allocation data store, an indication of a selected requested resource allocation between the enterprise and an entity; means for retrieving, by the back-end application computer server from a resource allocation data store, an electronic record associated with the selected requested resource allocation, including the set of resource allocation values associated with risk attributes, wherein the resource allocation data store contains electronic records that represent a plurality of requested resource allocations between the enterprise and a plurality of entities, and each electronic record includes an electronic record identifier and a set of resource allocation values associated with risk attributes that have been collected from the entities and service providers; and means for automatically determining an initial duration for the selected requested resource allocation based on the shortest of: (i) an entity expected duration, (ii) a service provider expected duration, and (iii) a third-party guideline expected duration.

In some embodiments, a communication device associated with a back-end application computer server exchanges information with remote devices in connection with an interactive graphical user interface associated with a workflow. The information may be exchanged, for example, via public and/or proprietary communication networks.

A technical effect of some embodiments of the invention is an improved and computerized way to provide an automated risk relationship resource allocation tool in a way that provides faster results in an improved way to ensure accuracy and consistency as compared to traditional approaches. With these and other advantages and features that will become hereinafter apparent, a more complete understanding of the nature of the invention can be obtained by referring to the following detailed description and to the drawings appended hereto.

### BRIEF DESCRIPTION OF THE DRAWINGS

FIG. 1 is a high-level block diagram of a system in accordance with some embodiments.

FIG. 2 illustrates a method according to some embodiments of the present invention.

FIG. 3 is a STD initial decision tool status indicator display in accordance with some embodiments.

FIG. 4 is a STD initial decision summary display according to some embodiments.

FIG. 5 is a STD initial decision tool notification display in accordance with some embodiments.

FIG. 6 is a STD initial decision summary display with rules having a status of “FAIL” according to some embodiments.

FIG. 7 is an updated STD initial decision tool status indicator display in accordance with some embodiments.

FIG. 8 is another STD initial decision summary display according to some embodiments.

FIG. 9 is a STD initial decision summary display in accordance with some embodiments.

FIG. 10 is a STD initial decision summary display with rules having a status of “OVER” (override) according to some embodiments.

FIG. 11 is a STD initial decision summary display with “ALL” rules in accordance with some embodiments.

FIG. 12 is a STD initial decision tool actions box according to some embodiments.

FIG. 13 is an updated STD initial decision tool status indicator display in accordance with some embodiments.

FIG. 14 is a maternity display according to some embodiments.

FIG. 15 is an introductory storyboard display in accordance with some embodiments.

FIG. 16 is another storyboard display according to some embodiments.

FIG. 17 is an example of an automatically generated STD approval letter in accordance with some embodiments.

FIG. 18 is a more detailed high-level block diagram of a system in accordance with some embodiments.

FIG. 19 illustrates a method according to some embodiments of the present invention.

FIG. 20 is an algorithm to determine an initial duration setting in accordance with some embodiments.

FIG. 21 is an ICD code table according to some embodiments.

FIG. 22 is a method associated with a decision to expedite a STD insurance claim in accordance with some embodiments.

FIG. 23 is an initial extension analytical model process according to some embodiments.

FIG. 24 is more of the extension analytical model process in accordance with some embodiments.

FIG. 25 is an extension rules recommendation display according to some embodiments.

FIG. 26 is a master extension business rules table in accordance with some embodiments.

FIGS. 27A and 27B are an initial attending physician's statement form according to some embodiments.

FIG. 28 is an attending physician's statement rules table in accordance with some embodiments.

FIG. 29 is a cosmetic elective table according to some embodiments.

FIG. 30 is an operator or administrator display in accordance with some embodiments.

FIG. 31 is a block diagram of an apparatus in accordance with some embodiments of the present invention.

FIG. 32 is a portion of a tabular resource allocation database according to some embodiments.

FIG. 33 illustrates a tablet computer displaying a risk relationship resource allocation tool user interface according to some embodiments.

FIG. 34 illustrates an overall process in accordance with some embodiments.

### DETAILED DESCRIPTION

The present invention provides significant technical improvements to facilitate electronic messaging and dynamic data processing. The present invention is directed to more than merely a computer implementation of a routine or conventional activity previously known in the industry as it significantly advances the technical efficiency, access, and/or accuracy of communications between devices by implementing a specific new method and system as defined herein. The present invention is a specific advancement in the area of electronic risk analysis and/or resource allocation by providing benefits in data accuracy, data availability, and data integrity and such advances are not merely a longstanding commercial practice. The present invention provides improvement beyond a mere generic computer implementation as it involves the processing and conversion of significant amounts of data in a new beneficial manner as well as the interaction of a variety of specialized client and/or provider systems, networks, and subsystems. For example, in the present invention information may be processed, updated, and analyzed via a back-end-end application server to accurately improve the analysis of risk, the allocation of resources, and the exchange of information, thus improving the overall efficiency of the system associated with message storage requirements and/or bandwidth considerations (e.g., by reducing the number of messages that need to be transmitted via a network). Moreover, embodiments associated with collecting accurate information might further improve risk values, predictions of risk values, allocations of resources, the automatic communication of information to entities, electronic record processing decisions, etc.

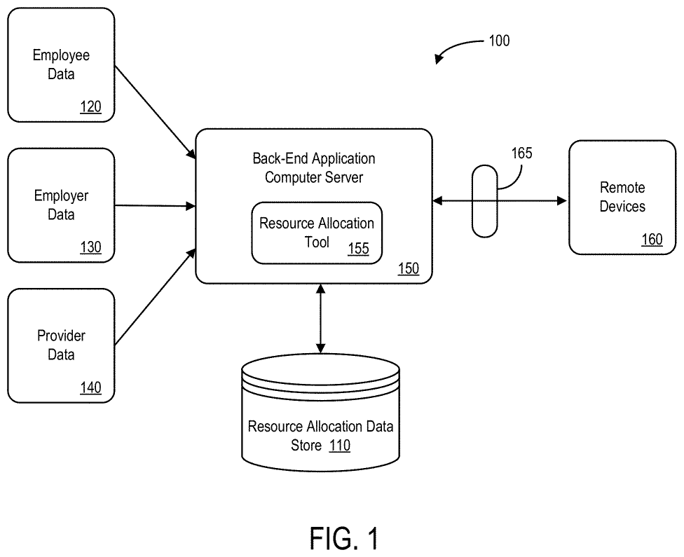

FIG. 1 is a high-level block diagram of a system 100 according to some embodiments of the present invention. In particular, the system 100 includes a back-end application computer server 150 that may access information in a resource allocation data store 110 (e.g., storing a set of electronic records representing requests for an allocation of resources, each record including, for example, one or more requested electronic record identifiers, attribute variables, resource values, etc.). The back-end application computer server 150 may also retrieve information from other data stores or sources (e.g., employee data 120 collected via a website or telephone call, employer data 130 such as Human Resources (“HR”) payroll information, and/or provider data 140 such as in-house medical service provider information, physician treatment information, or data from a third-party service provider) in connection with a resource allocation tool 155 and apply machine learning or artificial intelligence algorithms and/or models to the electronic records. The back-end application computer server 150 may also exchange information with a remote device 160 (e.g., via communication port 165 that might include a firewall). According to some embodiments, an interactive graphical user interface platform of the back-end application computer server 150 (and, in some cases, the provider data 140) may facilitate the display of information associated with the resource allocation tool 155 via one or more remote computers (e.g., to enable a manual review of a resource allocation request and/or an automatically selected workflow) and/or the remote device 160 (e.g., associated with a claim handler or ability analyst). For example, the remote device 160 may receive updated information (e.g., new injury information) from the back-end application computer server 150. Based on the updated information, a user may review the data from the resource allocation data store 110 and make informed decisions about requests. Note that the back-end application computer server 150 and/or any of the other devices and methods described herein might be associated with a cloud-based environment and/or a third-party, such as a vendor that performs a service for an enterprise.

The back-end application computer server 150 and/or the other elements of the system 100 might be, for example, associated with a Personal Computer (“PC”), laptop computer, smartphone, an enterprise server, a server farm, and/or a database or similar storage devices. According to some embodiments, an “automated” back-end application computer server 150 (and/or other elements of the system 100) may facilitate updates of electronic records in the resource allocation data store 110. As used herein, the term “automated” may refer to, for example, actions that can be performed with little (or no) intervention by a human.

As used herein, devices, including those associated with the back-end application computer server 150 and any other device described herein may exchange information via any communication network which may be one or more of a Local Area Network (“LAN”), a Metropolitan Area Network (“MAN”), a Wide Area Network (“WAN”), a proprietary network, a Public Switched Telephone Network (“PSTN”), a Wireless Application Protocol (“WAP”) network, a Bluetooth network, a wireless LAN network, and/or an Internet Protocol (“IP”) network such as the Internet, an intranet, or an extranet. Note that any devices described herein may communicate via one or more such communication networks.

The back-end application computer server 150 may store information into and/or retrieve information from the resource allocation data store 110. The resource allocation data store 110 might, for example, store electronic records representing a plurality of resource allocation requests, each electronic record having a set of attribute values including one or more resource values. The resource allocation data store 110 may also contain information about prior and current interactions with entities, including those associated with the remote devices 160. The resource allocation data store 110 may be locally stored or reside remote from the back-end application computer server 150. As will be described further below, the resource allocation data store 110 may be used by the back-end application computer server 150 in connection with an interactive user interface to provide information about the resource allocation tool 155. Although a single back-end application computer server 150 is shown in FIG. 1, any number of such devices may be included. Moreover, various devices described herein might be combined according to embodiments of the present invention. For example, in some embodiments, the back-end application computer server 150 and the resource allocation data store 110 might be co-located and/or may comprise a single apparatus.

According to some embodiments, the system 100 may be associated with Short Term Disability (“STD”) insurance claim files. As used herein, the phrase “STD insurance” might refer to insurance that insures a person's earned income against the risk that a disability creates a barrier for completion of core work functions. Elements of the system 100 might help, for example, an insurance claim handler or ability analyst quickly determine key claim information about an injured worker, insured, and/or treatment provider along with an appropriate workflow that can be used to help process the claim after a full and fair investigation and thorough review of the claim. Note that some workflows may involve a more detailed manual review as compared to other workflows (e.g., more straightforward claims).

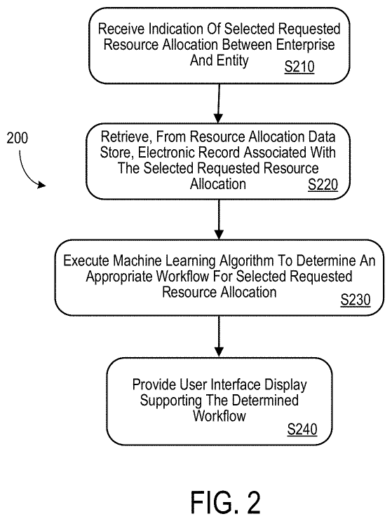

Note that the system 100 of FIG. 1 is provided only as an example, and embodiments may be associated with additional elements or components. According to some embodiments, the elements of the system 100 automatically transmit information associated with an interactive user interface display over a distributed communication network. FIG. 2 illustrates a method 200 that might be performed by some or all of the elements of the system 100 described with respect to FIG. 1, or any other system, according to some embodiments of the present invention. The flow charts described herein do not imply a fixed order to the steps, and embodiments of the present invention may be practiced in any order that is practicable. Note that any of the methods described herein may be performed by hardware, software, or any combination of these approaches. For example, a computer-readable storage medium may store thereon instructions that when executed by a machine result in performance according to any of the embodiments described herein.

At S210, a back-end application computer server (e.g., associated with an enterprise such as an insurer) may receive an indication of a requested resource allocation between the enterprise and an entity (e.g., an employee of a client of the insurer). For example, a claims hander or ability analyst associated with the enterprise might select a resource allocation request from a list of pending resource allocation requests. According to some embodiments, the user may search for requests based on a claim number, a request type, a date associated with the request, etc. At S220, the back-end application computer server may retrieve, from a resource allocation data store, an electronic record associated with the selected requested resource allocation, including the set of resource allocation values associated with risk attributes.

At S230, the system may execute a machine learning trained data science model to determine an appropriate workflow for the selected requested resource allocation. As used herein, the phrase “machine learning” may refer to various artificial intelligence techniques including algorithms and mathematical models that computer systems use to continuously improve performance associated with a specific task. Machine learning algorithms may comprise a mathematical model of sample data (e.g., “training data” associated with prior insurance claims) to make predictions. The machine learning algorithms and models might be associated with computational statistics, mathematical optimization, data mining, and/or predictive analytics. Moreover, the term “workflow” may refer to any process or template that might be used to evaluate an insurance claim (e.g., can review of the claim be expedited?).

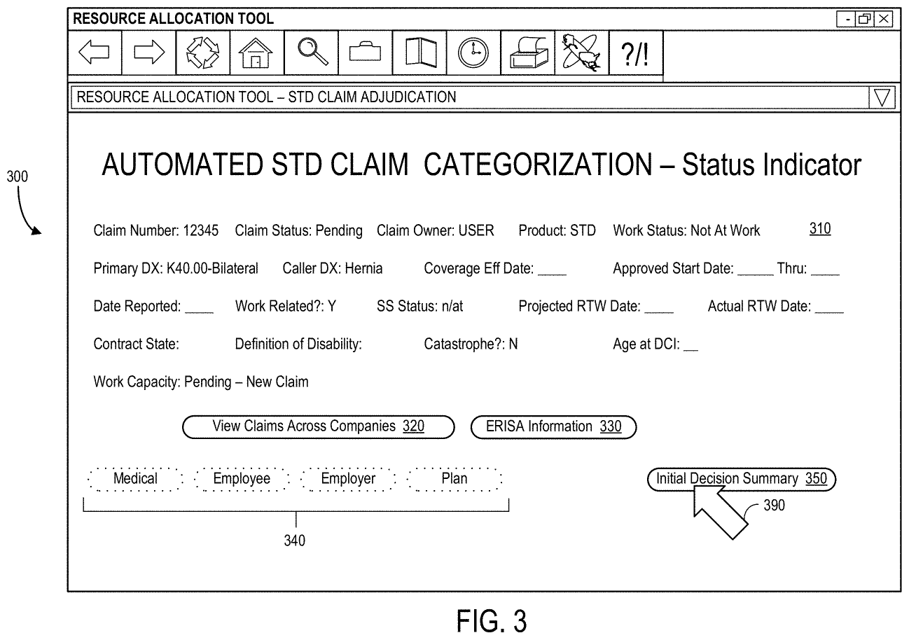

At S240, the system may provide user interface displays supporting the determined workflow. For example, an ability analyst may spend time reviewing and comparing fields across various screens to make an initial decision regarding a STD insurance claim. This might include opening a specific claim file and mapping a plan for the claim. FIG. 3 is a STD initial decision tool status indicator display 300 in accordance with some embodiments. The display 300 includes claim information 310, such as a claim number, claim status, Return-To-Work (“RTW”) information, etc. The display also includes a “View Claims Across Company” icon 320 and an “Employee Retirement Income Security Act (“ERISA”) information” icon 330. As will be described, claim rule category indicators 340 may be provided for medical, employee, employer, and plan categories.

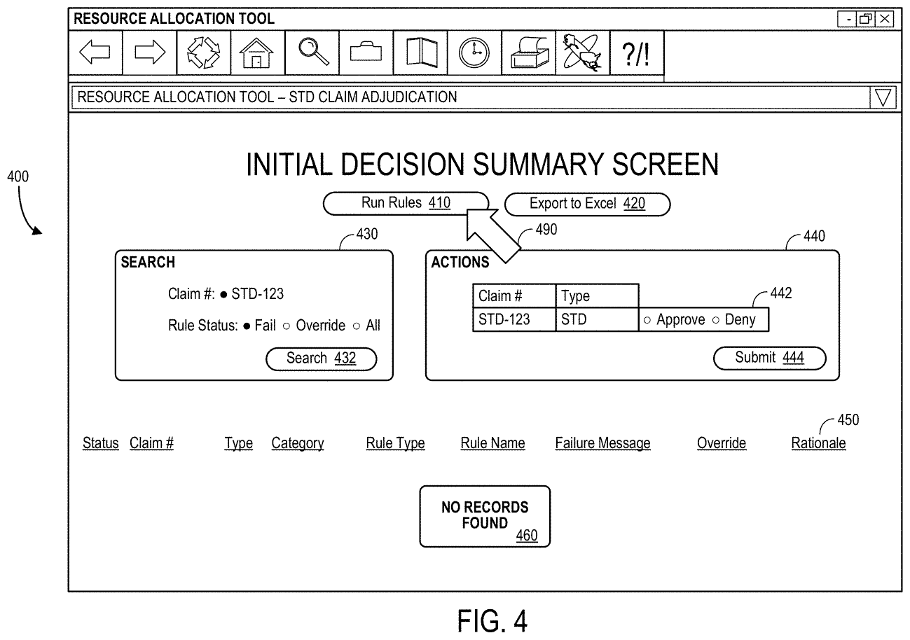

Selection of an “Initial Decision Summary” icon 350 (e.g., via touchscreen or computer mouse pointer 390) results in the display of an STD initial decision summary display 400, such as the one illustrated in FIG. 4 according to some embodiments. The initial decision summary display 400 includes a “Run Rules” icon 410 and an “Export to EXCEL®” icon 420 (e.g., to save information in a format suitable for a spreadsheet application). The initial decision summary display 400 further includes a search box 430 (with a “Search” icon 432) and an actions box 440 (with a claim table 442 and a “Submit” icon 444). A rule area 450 provides rule results, such as a rule status, a claim number, a claim type, a rule category, a rule name, etc. (and indicates that no records are currently found 460).

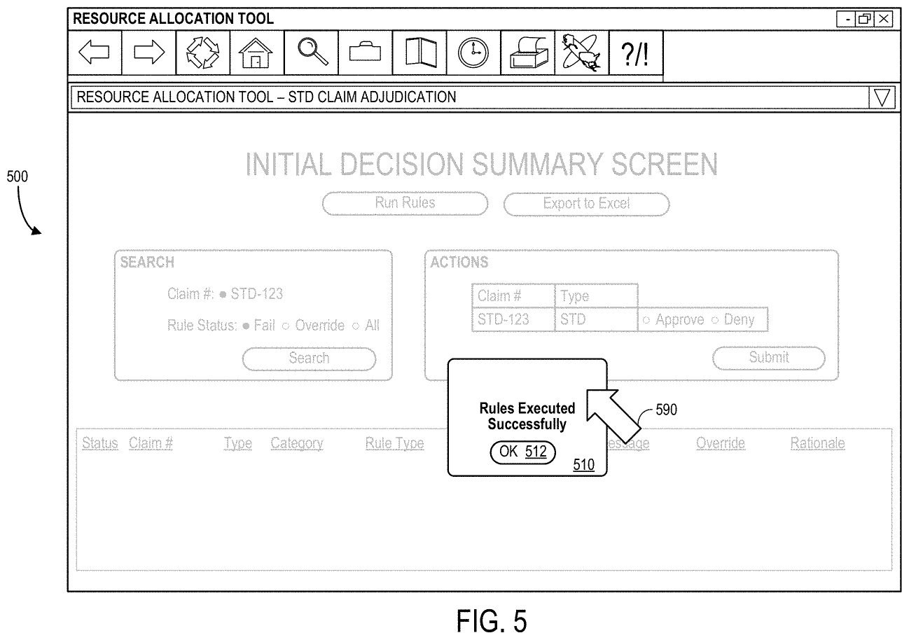

Selection of the “Run Rules” icon 410 (via a touchscreen or mouse pointer 490) results in display of a STD initial decision tool notification display 500 with a “Rules Executed Successfully” message 510 (including) an “OK” icon 512) as illustrated in FIG. 5. Selection of the “OK” icon 512 (via mouse pointer 590) results in display of a STD initial decision summary display 600 with rules having a status of “FAIL” according to some embodiments. In this case, the display 600 includes a rule area 650 showing that two rules have a status of “FAIL” (a missing hospital name and a missing emergency room statement date). A “Save” icon 660 can be used to store any adjustments to the rules. Note that according to some embodiments, the system executes “missing information” rules first. If there are any missing information rule failures, the analyst can address the failures and rerun the rules. If the analyst does not currently have the needed information, he or she can wait until it is received, update the claim, and rerun the rules.

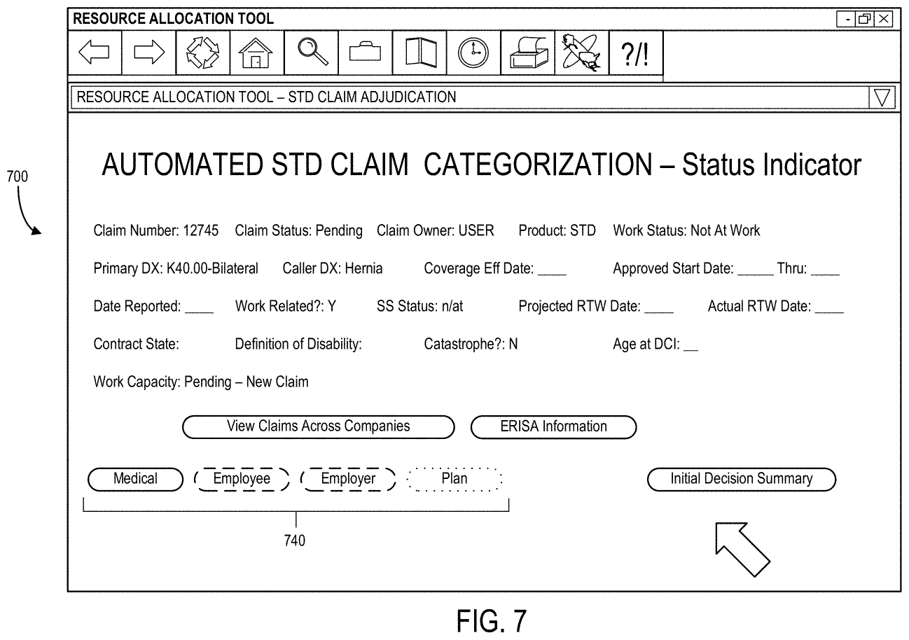

Next, the analyst can address any additional failures (that is, other than missing information) and rerun the rules. For example, FIG. 7 is an updated STD initial decision tool status indicator display 700 in accordance with some embodiments. Here, claim rule category indicators 740 have been updated to indicate that medical category rules have all passed (solid line), plan category rules are missing information (dotted line), and employee and employer category “missing information rules” but still have other types of rule failures (dashed line). If a mouse pointer hovers over one of the categories, the display 700 will indicate if it is a pass, missing information, or other type of category rule failure.

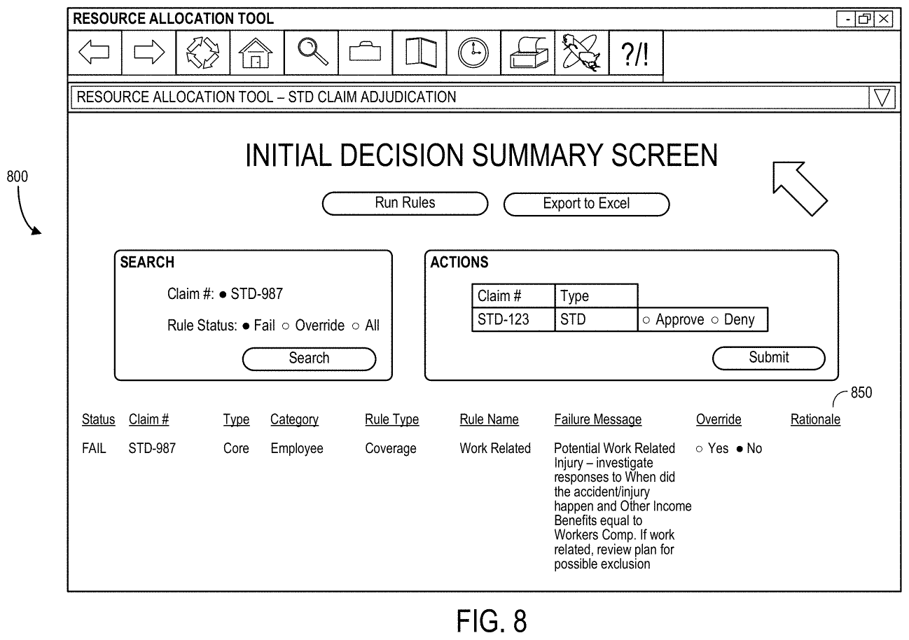

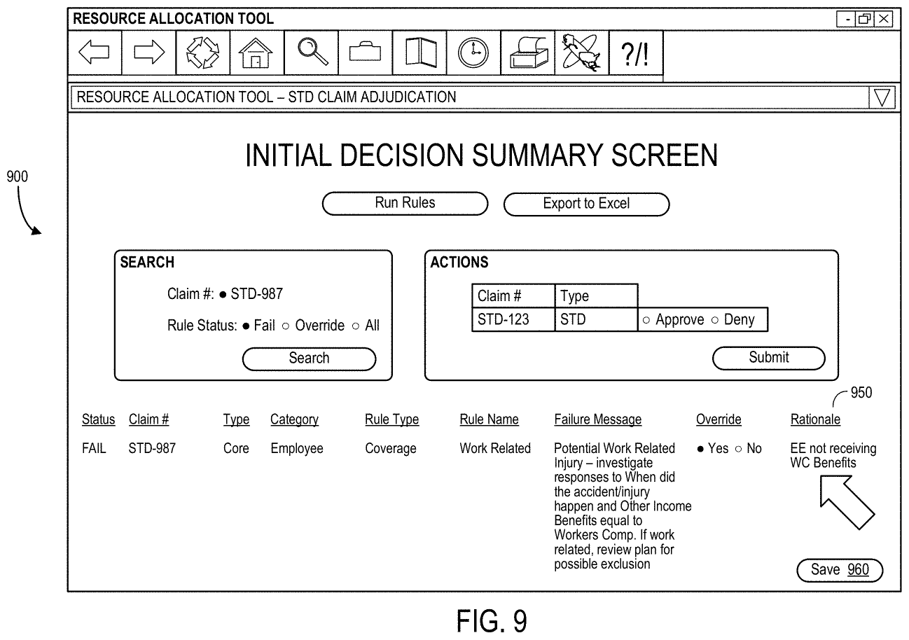

FIG. 8 is another STD initial decision summary display 800 with a rule area 850 showing that claim “STD-987” has failed a “coverage” rule in the employee category (for a potential work-related injury that might be covered by workers' compensation insurance). In this case, the analyst might add information to the claim (based on the failure message) or override the rule failure. For example, FIG. 9 is a STD initial decision summary display 900 in accordance with some embodiments. Here, a rule area 950 shows that the analyst has decided to override the failure and provided a rationale for the override (and this information may be stored using a “Save” icon 960). According to some embodiments, this rationale is part of the claim file so the content and/or rationale for the override should be concise (but make it easy to understand why the action was taken).

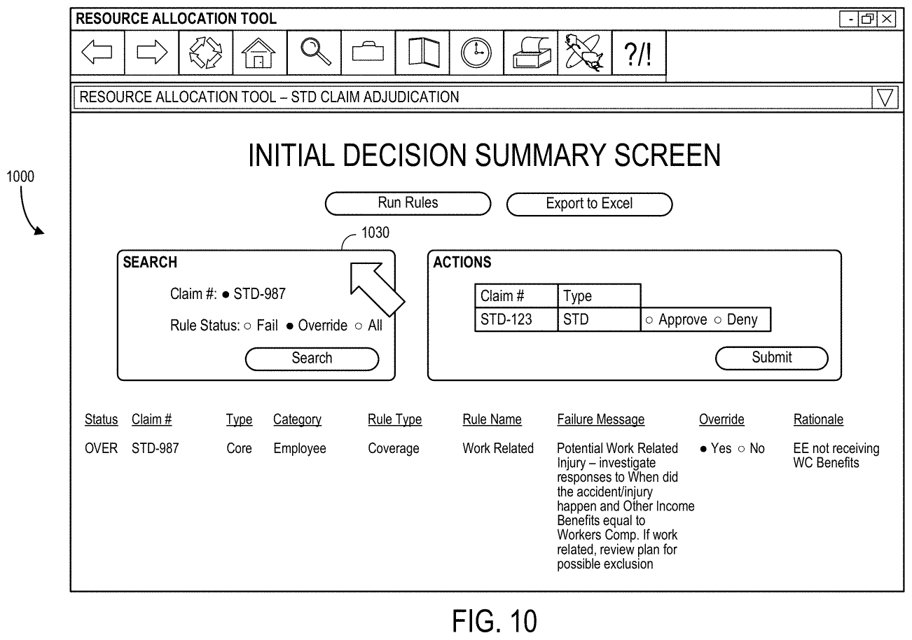

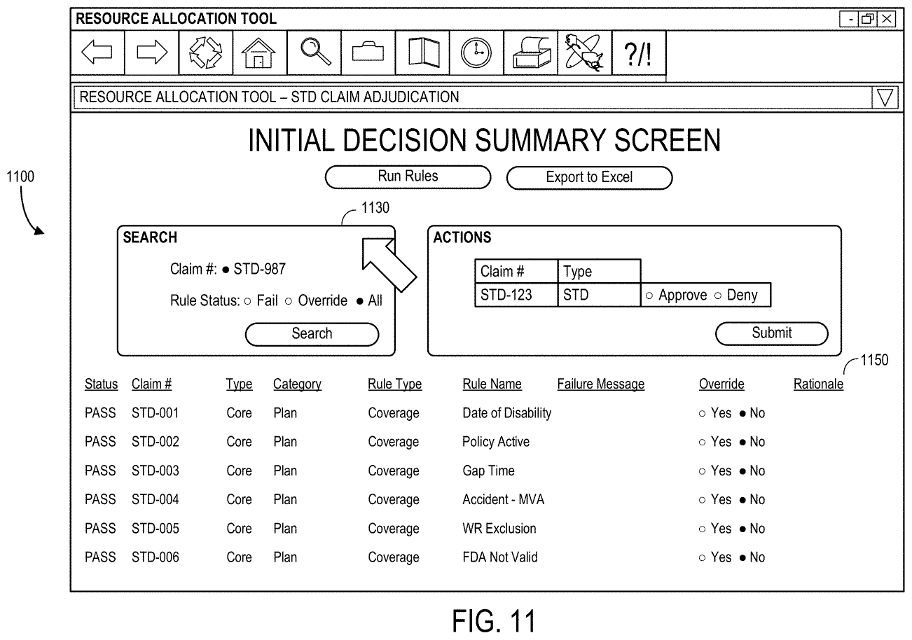

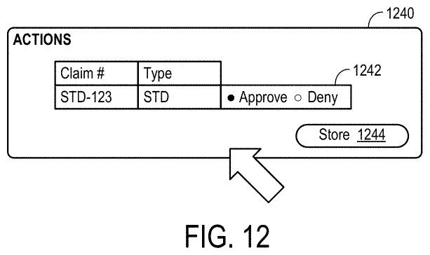

FIG. 10 is a STD initial decision summary display 1000 with rules having a status of “OVER” (override) according to some embodiments. In particular, a search box 1030 was used to search for rules with an “Override” status. Similarly, FIG. 11 is a STD initial decision summary display 1100 with a search box 1130 was used to display “All” rules in the rule area 1150 in accordance with some embodiments (including rules that have passed). After all of the rules pass, the analyst can review and approve the claim if the analyst determines that the claim meets approval requirements. For example, FIG. 12 is a STD initial decision tool actions box 1240 according to some embodiments. The box 1240 includes a claim table 1242 (e.g., to manually approve or deny the claim) and a “Store” icon 1244 that can be used to store the approval.

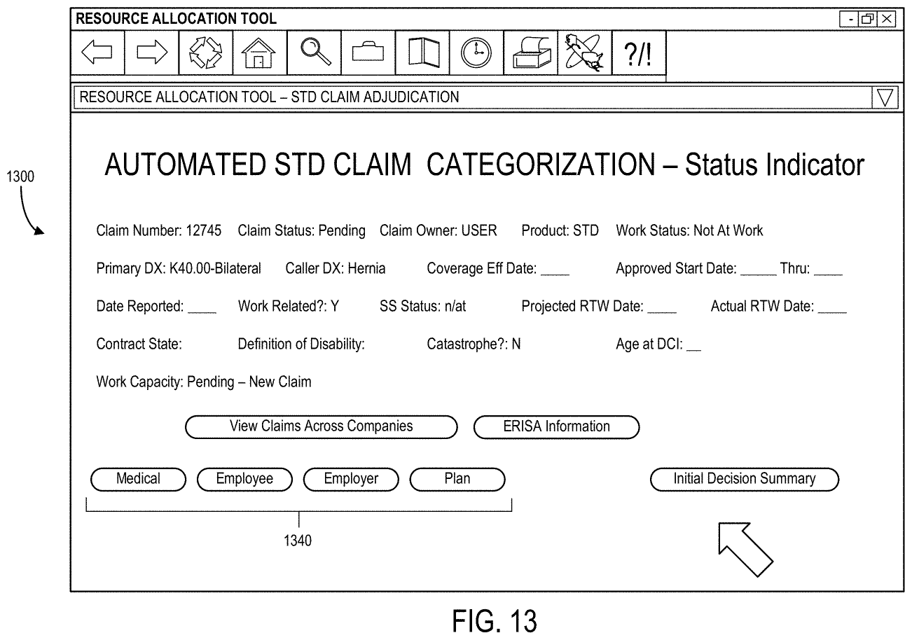

Storage of an approved claim may, in some embodiments, generate a process approval task assigned to an automated process (e.g., an automated process that from 7:00 am EST to 11:00 pm EST. The automated process may, for example, for this task and complete all of the administrative steps that are part of the claim decision, such as ensuring that a correct Date Of Diagnosis (“DOD”) is being used, calculating a claim duration, handling Leave Of Absence (“LOA”) situations, automatically generating and transmitting a message to the claimant, etc. FIG. 13 is an updated STD initial decision tool status indicator display 1300 with claim rule category indicators 1340 indicating that all category rules have all passed all rules (solid line).

An analyst might decide to execute rules in several different situations, such as:
upon an initial review of a new claim intake after the plan has been mapped;
anytime the analyst works a pending claim;
anytime the analyst overrides one or more rules;
after receiving information and/or updated data fields; and
when the analyst is ready to approve a claim, he or she might re-run the rules one last time.

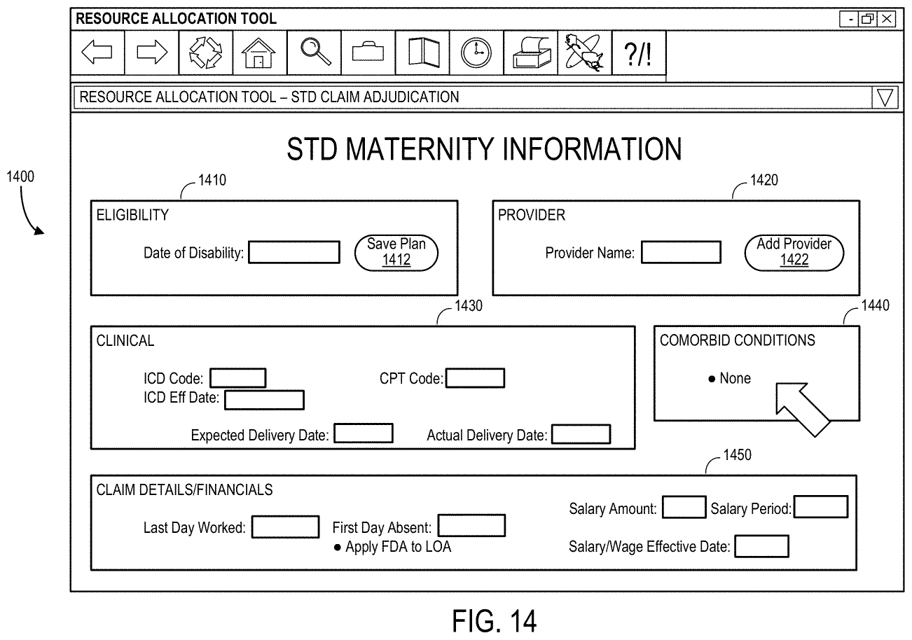

FIG. 14 is a maternity display 1400 according to some embodiments. When maternity information is received, the analyst may update various fields as needed: a date disability in an eligibility box 1410 (requires selection of a “Save Plan” icon 1412); a provider name in a provider box 1420 (requires selection of an “Add Provider” icon 1422), clinical codes in a clinical box 1430, selections in a comorbid conditions box 1440; and a last day worked and First Day Absent (“FDA”) in a claim details/financials box 1450.

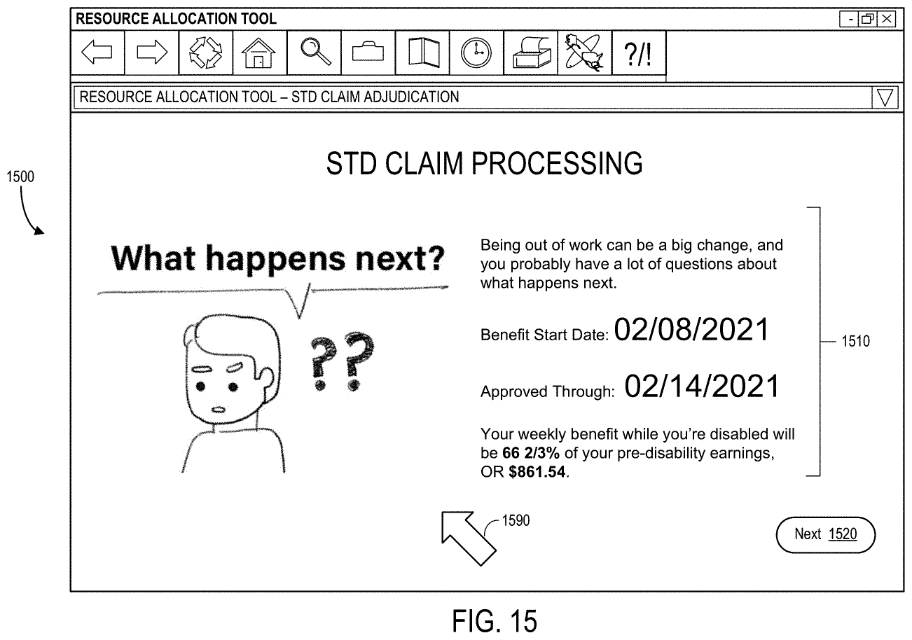

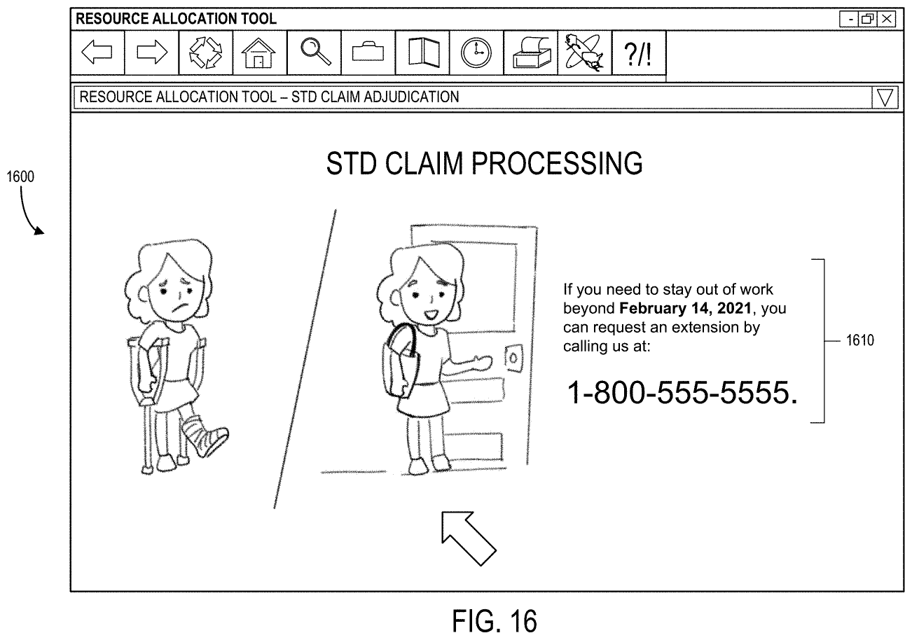

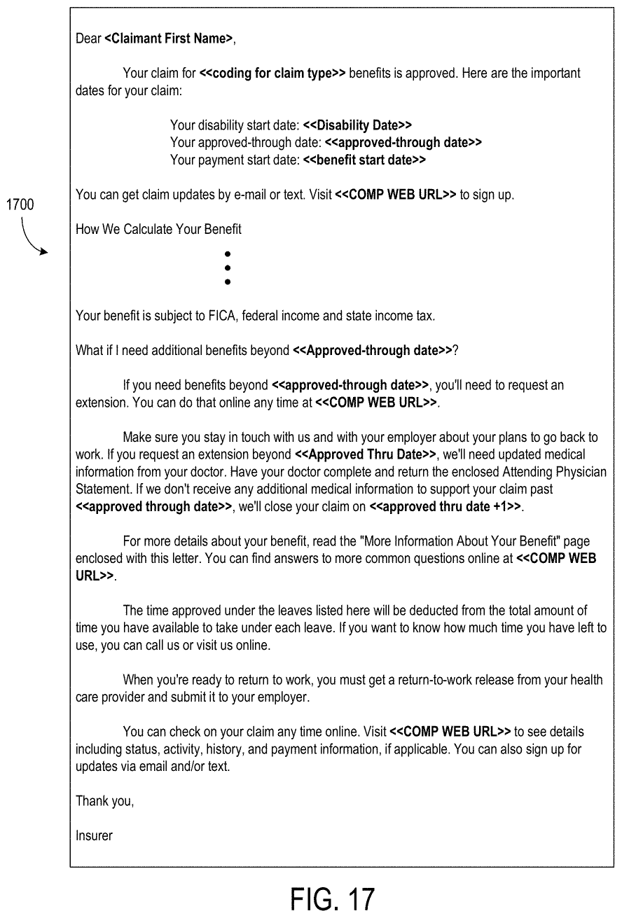

FIG. 15 is an introductory storyboard display 1500 that might be provided to a claimant when a STD insurance claim is approved in accordance with some embodiments. The storyboard may include graphics (including animations) and a description 1510 of benefit details (e.g., a benefit start date and dollar value). Selection of a “Next” icon 1520 (e.g., via a computer mouse pointer 1590) may provide another storyboard display 1600 as illustrated in FIG. 16 according to some embodiments. This display 1600 may also include graphics and a description 1610 of how to contact the insurer to request a STD insurance benefit extension. According to some embodiments, the storyboard displays 1500, 1600 may be associated with a customized video that is automatically created for and/or communicated to a particular claimant on-the-fly and in substantially real time (e.g., within hours or a few days of a workers' compensation claim being submitted). The displays 1500, 1600 may be generated using rules and logic to tailor the explanation so that it is appropriate for the claimant's situation. In addition to these types of storyboard displays 1500, 1600, in some embodiments the system also automatically generates a customized approval letter or message, such as the one 1700 illustrated in FIG. 17, to explain STD insurance benefits.

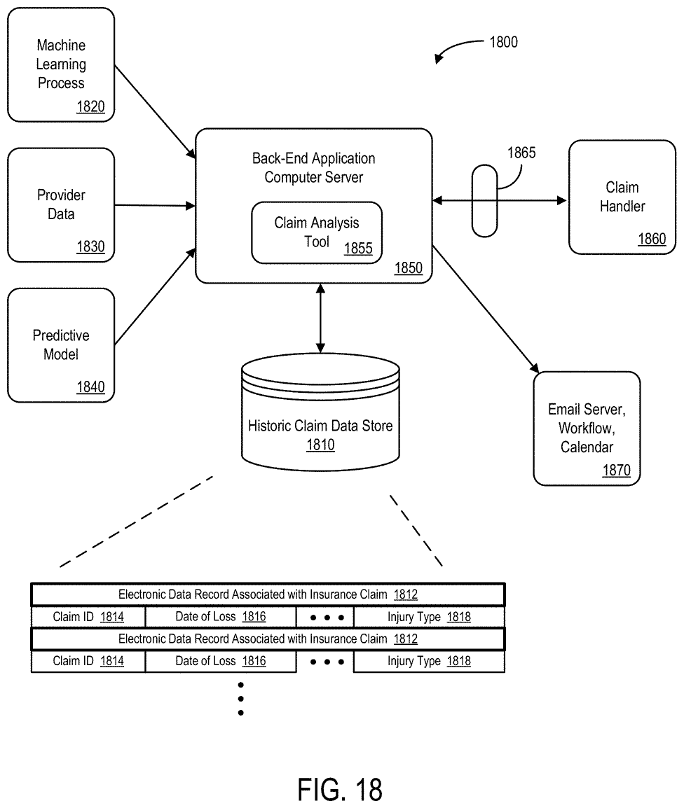

FIG. 18 is a more detailed high-level block diagram of a system 1800 in accordance with some embodiments. As before, the system 1800 includes a back-end application computer server 1850 that may access information in a historic claim data store 1810. The back-end application computer server 1850 may also retrieve information from a machine learning process 1820, provider data 1830 (e.g., from medical providers or police reports), and/or predictive models 1840 in connection with an insurance claim analysis tool 1855. The back-end application computer server 1850 may also exchange information with a claim handler device 1860 (e.g., via communication port 1865 that might include a firewall) to enable a manual review of a STD insurance claim. The back-end application computer server 1850 might also transmit information directly to an email server (or postal mail server), a workflow application, and/or a calendar application 1870 to facilitate STD insurance claim processing.

The back-end application computer server 1850 may store information into and/or retrieve information from the historic claim data store 1810. The historic claim data store 1810 might, for example, store electronic records 1812 representing a plurality of STD insurance claims (e.g., resource allocation requests), each electronic record having a set of attribute values including a claim identifier 1814, a date of loss 1816, an injury type 1818, etc. According to some embodiments, the system 1800 may also provide a dashboard view of STD insurance claim files.

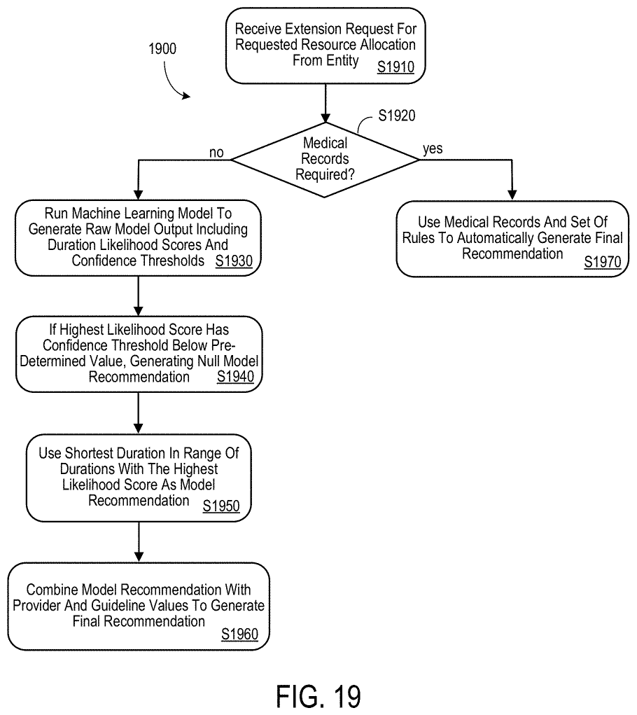

FIG. 19 illustrates a method 1900 according to some embodiments of the present invention. At S1910, a back-end application computer server (e.g., associated with an insurer) may receive an extension request for a requested resource allocation from an entity. For example, if STD benefits were originally approved for one month, an employee might request that STD benefits be extended to last six weeks. If it is determined at S1920 that medical records (or other service provider records) are not required (e.g., the claim is relatively common and does not need additional documentation), the system may execute a Machine Learning (“ML”) model to generate raw model output including duration likelihood scores (e.g., as illustrated in Table 1) and confidence thresholds (e.g., as illustrated in Table 2) at S1930.

TABLE 1
Raw Model Output - Duration Likelihood Scores (Days)

| 10-15 | 15-22 | 22-30 | 30-40 | 40-47 | 47-61 | 61-86 |
|---|---|---|---|---|---|---|
| 3% | 4% | 6% | 6% | 13% | 47% | 11% |

TABLE 2
Raw Model Output - Confidence Thresholds

| 10-15 | 15-22 | 22-30 | 30-40 | 40-47 | 47-61 | 61-86 |
|---|---|---|---|---|---|---|
| 0.11 | 0.11 | 0.11 | 0.11 | 0.11 | 0.21 | 0.23 |

If the highest likelihood score (e.g., 47-61 days in Table 1) has a confidence threshold below a pre-determined value at S1940, the system may generate a “null” model recommendation. At S1950, the system may use the shortest duration in the range of durations (e.g., with 47 days being the shortest duration in the range “47-61” illustrated in Table 1) as the model recommendation. The system may then combine the model recommendation with provider and guideline values to generate a final recommendation at S1960. If it was instead determined at S1920 that medical records (or other service provider records) are required (e.g., the claim is relatively uncommon and/or especially serious), the system may use the medical records and a set of rules to automatically generate a final recommendation at S1970.

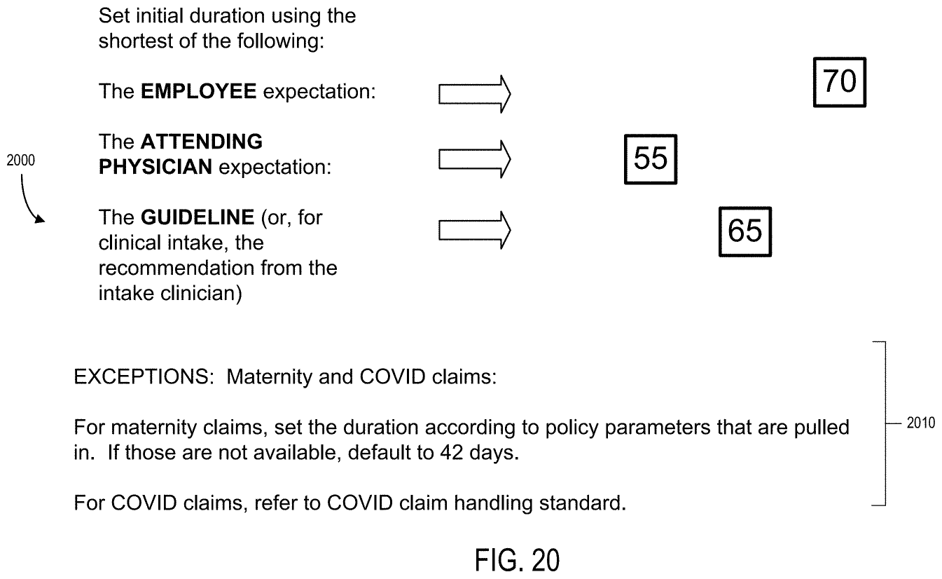

FIG. 20 is an algorithm 2000 to determine an initial duration setting in accordance with some embodiments. In particular, the algorithm 2000 may set the initial duration using the shortest of the following values: an employee expectation, an attending physician expectation, and a guideline value such as an Official Diagnostic Guideline (“ODG”) value based on past STD claims. The algorithm 2000 may also process exceptions 2010, such as those associated with maternity leave, COVID claims, etc. As illustrated in FIG. 20, “55” days may be used as the initial duration setting (as being the shortest duration).

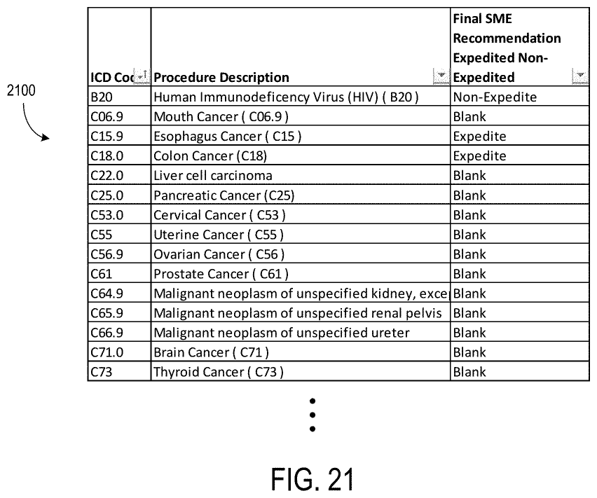

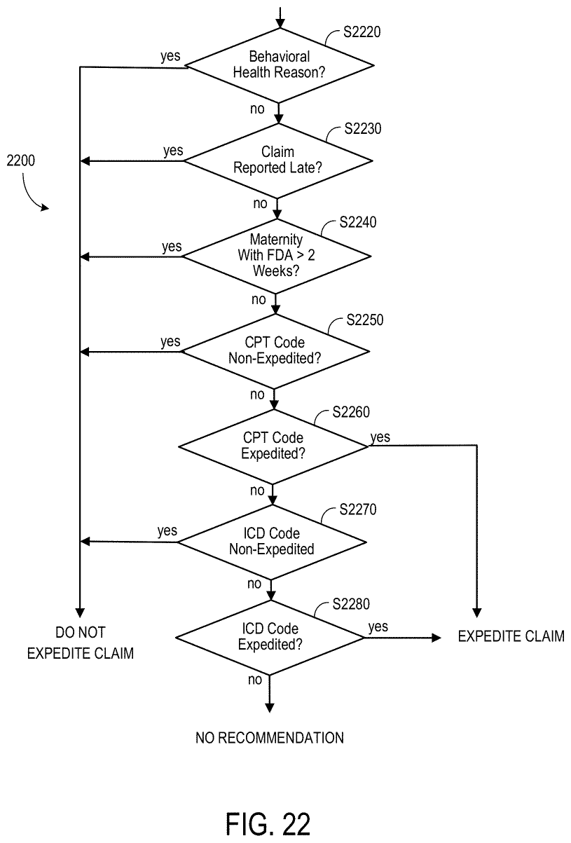

FIG. 21 is an International Classification of Diseases (“ICD”) code table 2100 according to some embodiments. The ICD are codes used in patient paperwork (e.g., hospital records, medical charts, visit summaries, bills, etc.). Current Procedural Terminology (“CPT”) codes are five-digit number that are similar in nature to ICD codes. These codes can help patients receive appropriate treatment and medical services. FIG. 22 is a method 2200 associated with a decision to expedite a STD insurance claim in accordance with some embodiments. If it is determined at S2220 that a claim is associated with a behavioral health reason, a “do not expedite claim” output is generated. If it is determined at S2230 that a claim was reported late (e.g., greater than 45 days and RTW=“no”), a “do not expedite claim” output is generated. If it is determined at S2240 that it is associated with a maternity claim and the FDA is greater than 2 weeks, a “do not expedite claim” output is generated. If it is determined at S2250 that a CPT code associated with the claim is marked as “non-expedited,” a “do not expedite claim” output is generated. If it is determined at S2260 that the CPT code is marked as “expedited,” an “expedite claim” output is generated. If it is determined at S2270 that an ICD code associated with a claim is marked as “non-expedited,” a “do not expedite claim” output is generated. If it is determined at S2280 that the ICD code is marked as “expedited,” a “do expedite claim” output is generated. If none of the checks in S2220 through S2280 resulted in an output, the system will generate no recommendation.

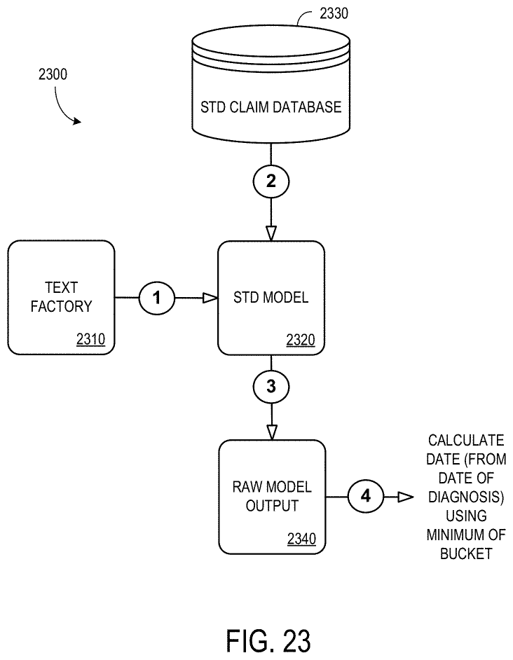

Embodiments may utilize an algorithm to recommend extension of benefits for an extension request without needing medical records. For example, approximately 50% short term disability extension requests might not require medical records. By quickly and accurately processing such requests, the system may be able to provide more satisfactory customer service. FIG. 23 is an initial extension analytical model process 2300 according to some embodiments. The process may begin at (1) by sending information from a text factory 2310 (e.g., that identifies and flags text in, for example, bill data in a bill review system) to an STD model 2320. This might include, for example, information associated with STD daily non-cancelled claims, specific types of claims to be dropped (e.g., maternity, particular ICD codes, etc.), old claims (e.g., more than one year from the DOD), claims that have been migrated, etc. This information may be used to filter data received from a STD claim database 2330 at (2). The STD model 2320 can then generate raw model output 2340 at (3), such as the information described in connection with Tables 1 and 2.

According to some embodiments, the functions performed in connection with (1), (2), and (3) in FIG. 23 may be executed and/or refreshed at pre-determined times each day. For example, the STD claim database 2330 may be refreshed daily at 8:00 AM, the text factory 2310 may be refreshed daily at 10:00 AM, and the STD model 2320 may be executed daily at 12:00 PM.

Note that in some embodiments, claim information might not be shared with certain customers (e.g., for privacy reasons). The highest scoring range of durations may then be selected (assuming the confidence level is high enough) and the date may be calculated at (4) from the DOD using the minimum number of days in the selected range of durations. If the raw model output 2340 does not pass the confidence threshold, the model recommendation is “null.” For example, if 47 days is the minimum number (as illustrated in Table 1) and the DOD was 6/1/2025, then the calculated date would be 6/1/2020+47 days=7/18/2025 assuming the raw model output was associated with a sufficient level of confidence.

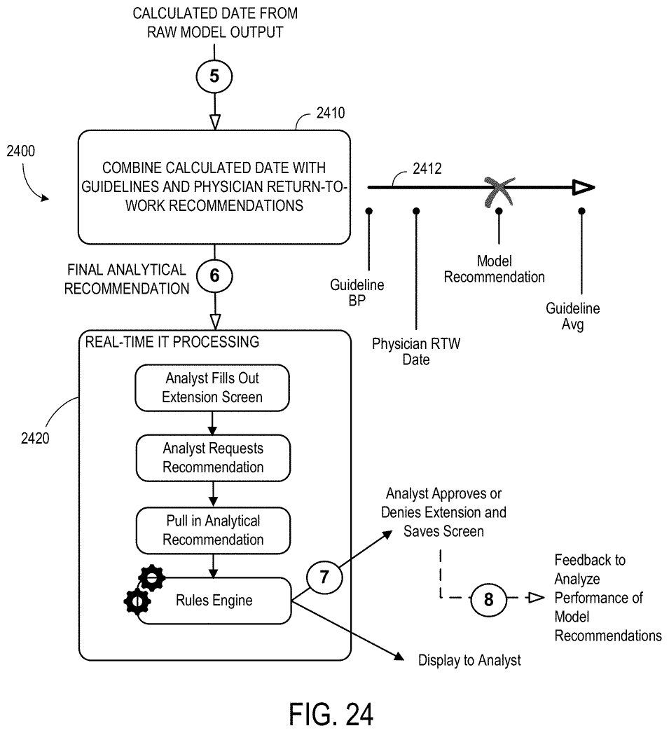

FIG. 24 is more of the extension analytical model process 2400 in accordance with some embodiments. The calculated date is received at (5) and combined with guideline and physician RTW recommendations 2410 as shown in a timeline 2412. If the model recommendation and physician RTW date are null, then ODG Best Practices (“BP”) date is used. Otherwise, the system may use the physician RTW date if present. If the physician RTW date is not present, the system may use the model recommendation. According to some embodiments, the final recommendation should never be less than ODG BP date or greater than the ODG average date. If the physician RTW date exceeds ODG average date, then the system may use the ODG BP date. This process produces the final analytical recommendation that is provided to real-time Information Technology (“IT”) processing 2420 at (6).

The real-time IT processing 2420 may involve an analyst filling out a user interface extension screen and selecting a “Get Recommendation” icon. The system may then pull the final analytical recommendation and execute a rules engine to display a result to the analyst and/or have the analyst manually approve or deny the extension at (7). Information may be saved back into the STD claim database 2330 and be used in a feedback loop at (8) to analyze performance of final analytical recommendation and improve the STD model 2320. In this way, the system may continuously adjust operation to achieve better performance. As a result, the system may provide more satisfactory customer service by quickly and accurately processing extension requests.

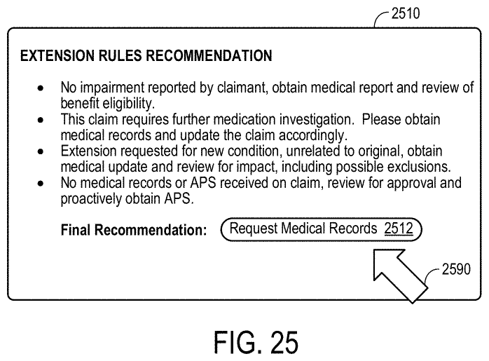

FIG. 25 is an extension rules recommendation display 2510 according to some embodiments. The display 2510 includes various rule messages, such as “Appropriate to Approve,” “Review without new Medical,” “Request updated Medical,” “Request updated Medical and consider clinical referral,” etc. The display 2510 also includes a final recommendation or action that might be, for example, a “Request Medical Records” icon 2512 that can be selected (e.g., via a touchscreen or computer mouse pointer 2590) to perform that action. In this way, an analyst can immediately and easily determine and provide an appropriate response to a STD extension request.

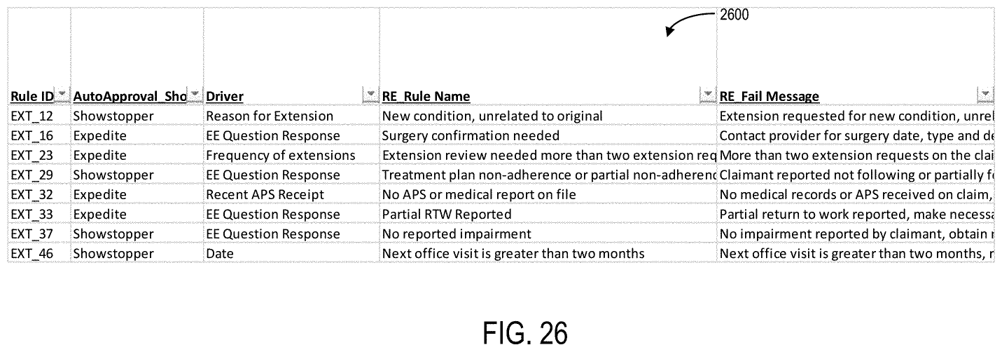

FIG. 26 is a master extension business rules table 2600 in accordance with some embodiments. For each rule, the table 2600 includes a rule identifier, an automatic approval showstopper value (e.g., indicating if the claim can be expedited), a rule drive (e.g., the reason for an extension, an employee question response, restrictions, policies, etc.), a rule name, and a failure message that might be displayed on the extension rules recommendation display 2510 when a claim fails a particular rule (e.g., “More than two extension requests on the claim”).

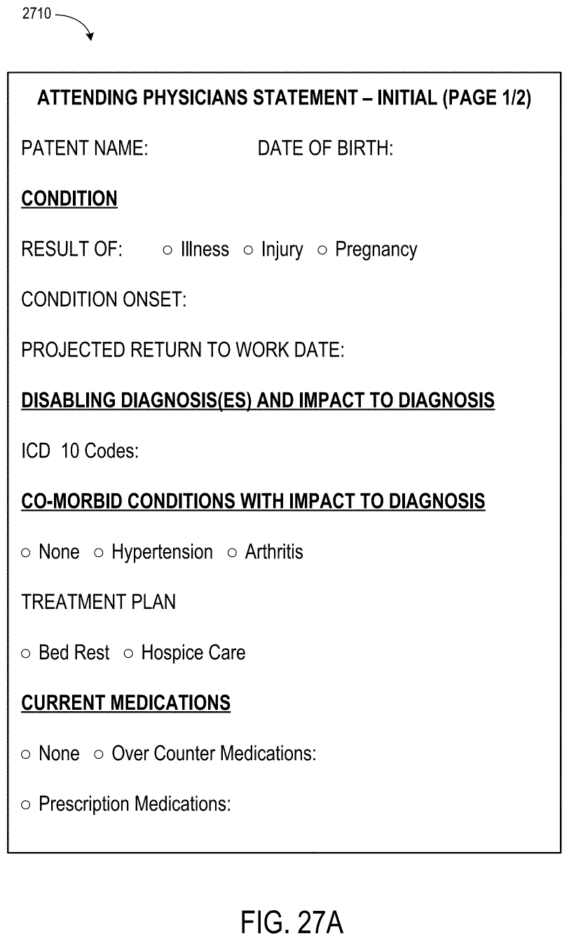

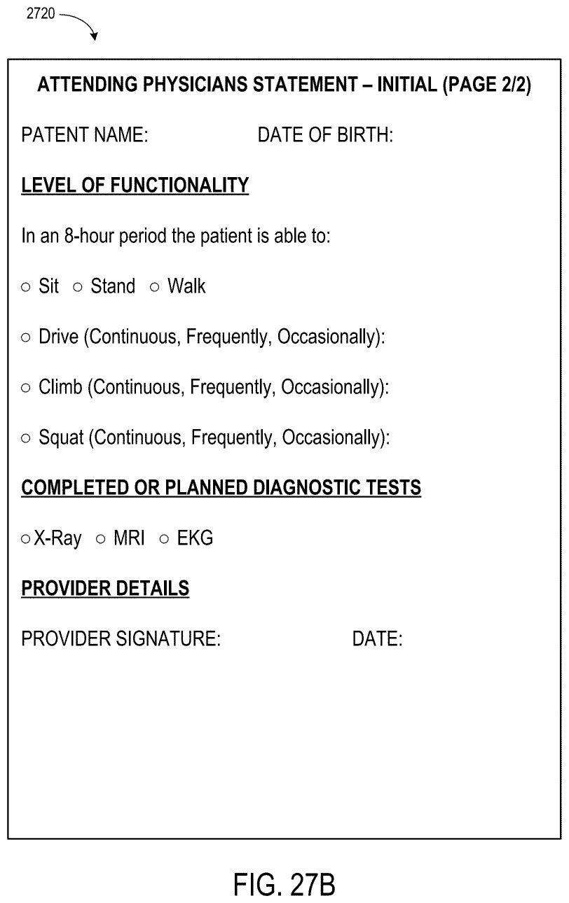

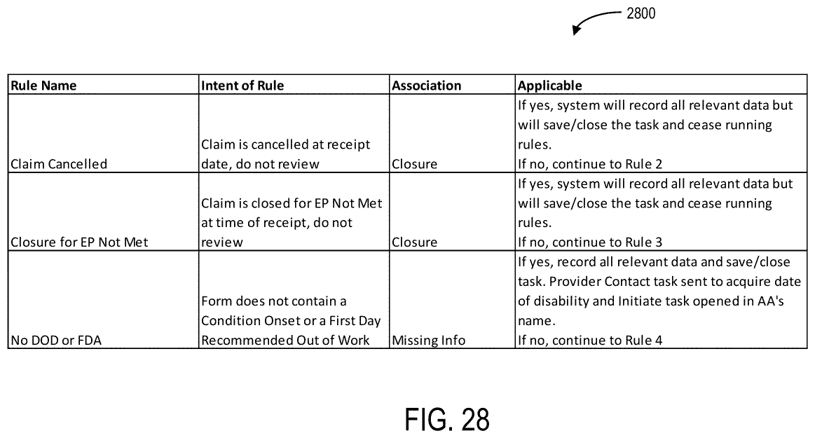

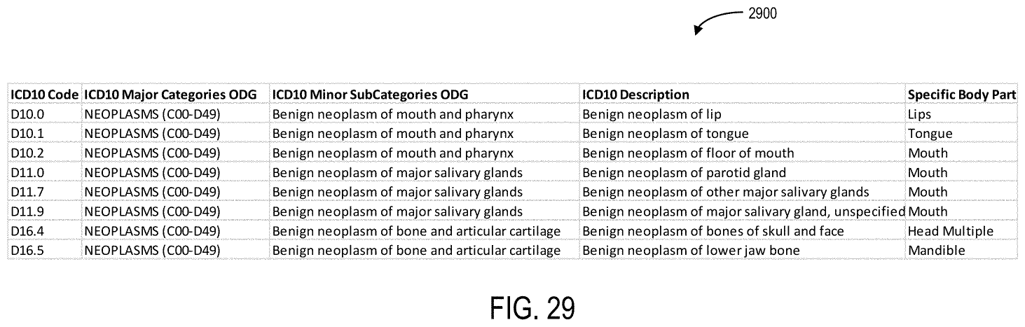

FIGS. 27A and 27B are an initial attending physician's statement form 2710, 2720 according to some embodiments. The form 2710, 2720 might include patient details (e.g., name and date of birth), condition information (e.g., a RTW date), any disabling diagnoses, co-morbidity conditions, treatment plan data, current medications, level of functionality information, completed or planned diagnostic tests, provider details (e.g., name and specialty), etc. The information in the initial attending physician's statement form 2710, 2720 may be used in connection with an attending physician's statement rules table 2800 as illustrated in FIG. 28 in accordance with some embodiments. The table 2800 includes a rule name, an intent of the rule, a rule association, and any further applicable details. Other tables may be associated with rules, such as a cosmetic elective table 2900 as illustrated in FIG. 29 according to some embodiments. The table 2900 includes an ICD code, categories and sub-categories, description, etc.

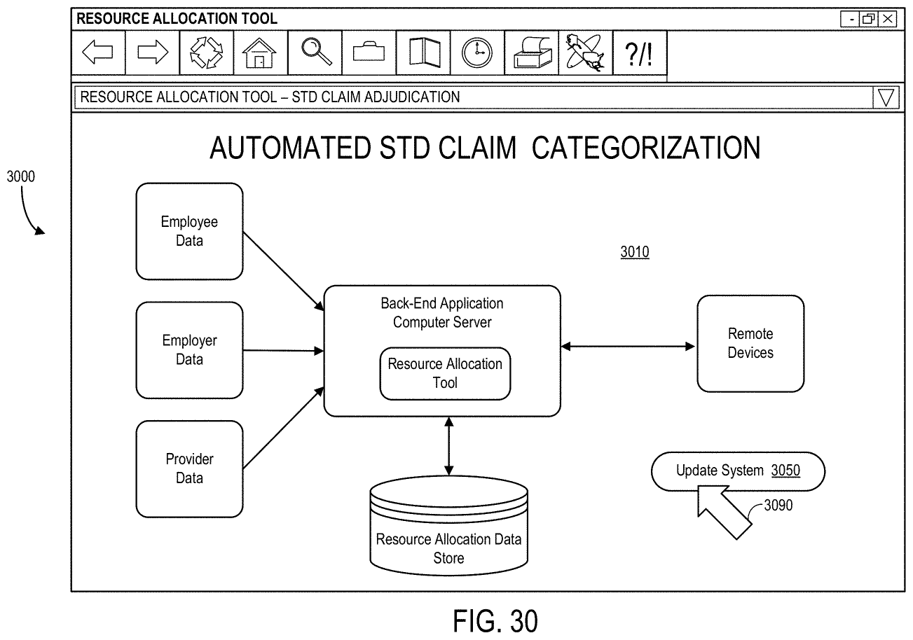

FIG. 30 is an operator or administrator display 3000 including graphical representations of elements of a STD claim categorization system 3010 in accordance with any of the embodiments described herein. Moreover, selection of an element, such as a computer server or data source (e.g., via touchscreen or computer mouse pointer 3090) may display configuration information about that element and/or let an operator or administrator adjust the configuration (e.g., to adjust model parameters). The display 3000 may further let the operator or administrator select an “Update System” icon 3050 to cause the system or platform to save changes, apply reconfigurations, etc.

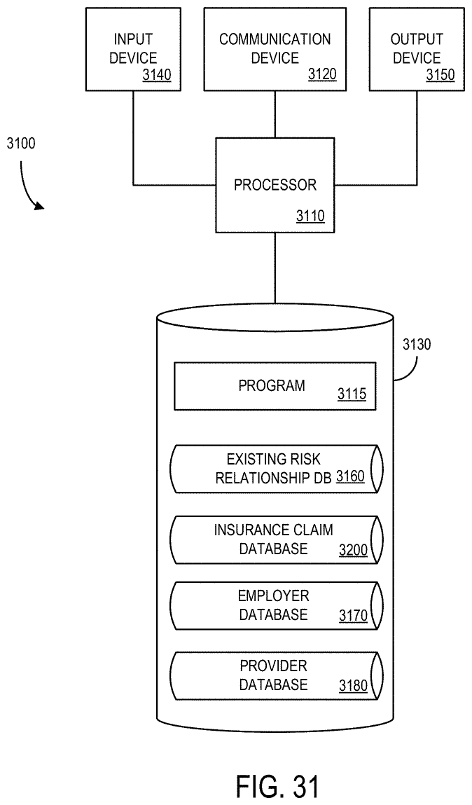

The embodiments described herein may be implemented using any number of different hardware configurations. For example, FIG. 31 illustrates an apparatus 3100 that may be, for example, associated with the systems 100, 1800 described with respect to FIGS. 1 and 18, respectively. The apparatus 3100 comprises a processor 3110, such as one or more commercially available Central Processing Units (“CPUs”) in the form of one-chip microprocessors, coupled to a communication device 3120 configured to communicate via communication network (not shown in FIG. 31). The communication device 3120 may be used to communicate, for example, with one or more remote claim handler and ability analyst computers and/or communication devices (e.g., PCs and smartphones). Note that communications exchanged via the communication device 3120 may utilize security features, such as those between a public internet user and an internal network of the insurance enterprise. The security features might be associated with, for example, web servers, firewalls, and/or PCI infrastructure. The apparatus 3100 further includes an input device 3140 (e.g., a mouse and/or keyboard to enter information about an employee, injuries, claim dates, etc.) and an output device 3150 (e.g., to output reports regarding STD insurance claim status).

The processor 3110 also communicates with a storage device 3130. The storage device 3130 may comprise any appropriate information storage device, including combinations of magnetic storage devices (e.g., a hard disk drive), optical storage devices, mobile telephones, and/or semiconductor memory devices. The storage device 3130 stores a program 3115 and/or a resource allocation tool or application for controlling the processor 3110. The processor 3110 performs instructions of the program 3115, and thereby operates in accordance with any of the embodiments described herein. For example, the processor 3110 may provide an automated risk relationship resource allocation tool. A resource allocation data store may contain electronic records representing requested resource allocations between the enterprise and a plurality of entities. The processor 3110 may receive an indication of a selected requested resource allocation and retrieve, from the resource allocation data store, the electronic record associated with the selected requested resource allocation. The processor 3110 may also execute a machine learning trained data science model to determine an appropriate workflow for the selected requested resource allocation. The processor 3110 may then support a graphical interactive user interface display via a distributed communication network, the interactive user interface display providing resource allocation data.

The program 3115 may be stored in a compressed, uncompiled and/or encrypted format. The program 3115 may furthermore include other program elements, such as an operating system, a database management system, and/or device drivers used by the processor 3110 to interface with peripheral devices.

As used herein, information may be “received” by or “transmitted” to, for example: (i) the apparatus 3100 from another device; or (ii) a software application or module within the apparatus 3100 from another software application, module, or any other source.

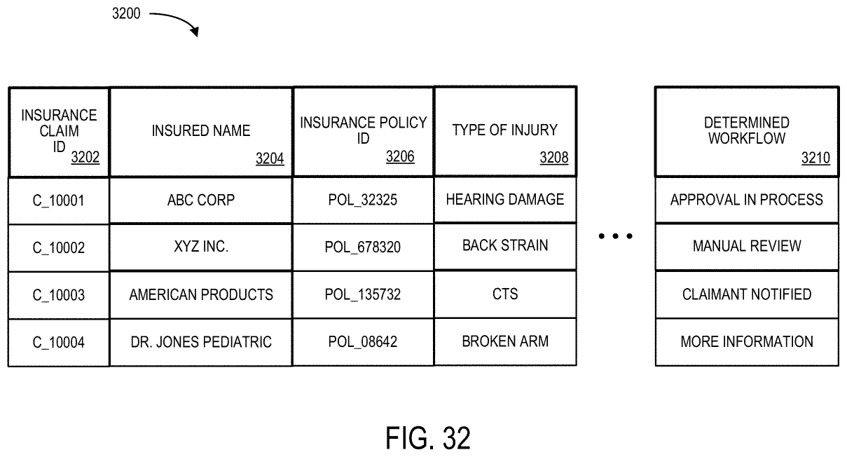

In some embodiments (such as shown in FIG. 31), the storage device 3130 further stores an existing risk relationship database 3160 (e.g., containing insurance policy information), an insurance claim database 3200, an employer database 3170, and a provider database 3180. An example of a database that might be used in connection with the apparatus 3100 will now be described in detail with respect to FIG. 32. Note that the database described herein is only an example, and additional and/or different information may be stored therein. Moreover, various databases might be split or combined in accordance with any of the embodiments described herein. For example, the existing risk relationship database 3160 and the insurance claim database 3200 might be combined and/or linked to each other within the program 3115.

Referring to FIG. 32, a table is shown that represents the insurance claim database 3200 that may be stored at the apparatus 3100 according to some embodiments. The table may include, for example, entries associated with STD insurance claims that have been submitted by claimants. The table may also define fields 3202, 3204, 3206, 3208, 3210 for each of the entries. The fields 3202, 3204, 3206, 3208, 3210 may, according to some embodiments, specify: an STD insurance claim identifier 3202, an insured name 3204, an insurance policy identifier 3206, a type of injury 3208, and a determined workflow 3210. The insurance claim database 3200 may be created and updated, for example, based on information electrically received from various operators, administrators, and computer systems, including those associated with an insurer.

The insurance claim identifier 3202 may be, for example, a unique alphanumeric code identifying a request for resources (e.g., when an employee working for an insured becomes injured). The insured name 3204 might be associated with the owner of insurance policy associated with the insurance policy identifier 3206. The type of injury 3208 might indicate when the employee was hurt. Note that the database 3200 might include additional information about each STD insurance claim (not illustrated in FIG. 32), such as claim handler notes, medical treatment costs, legal negotiations, etc. The determined workflow 3210 might indicate that the STD insurance claim is going through an approval process, a manual review, whether or not a claimant has been notified about a decision, etc.

Thus, embodiments may provide an automated and efficient process to provide an automated risk relationship resource allocation tool in a way that provides faster results in an improved way to ensure accuracy and consistency as compared to traditional approaches. Embodiments may aggregate data from multiple sources and use data science models to help claim handlers quickly recognize which claims might need closer attention. By digesting information, such as medical records, and applying artificial intelligence, embodiments may leverage available data and automate medical approval judgements, help motivate and influence STD claimant behavior, reduce a number of electronic records that need to be transmitted via communication networks, etc.

The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although specific hardware and data configurations have been described herein, note that any number of other configurations may be provided in accordance with embodiments of the present invention (e.g., some of the databases described herein may be combined or stored in external systems). Moreover, although embodiments have been described with respect to particular types of insurance policies, embodiments may instead be associated with other types of insurance policies in additional to and/or instead of the policies described herein. Similarly, although certain attributes (e.g., values analyzed in connection with resource allocation requests) were described in connection some embodiments herein, other types of attributes might be used instead.

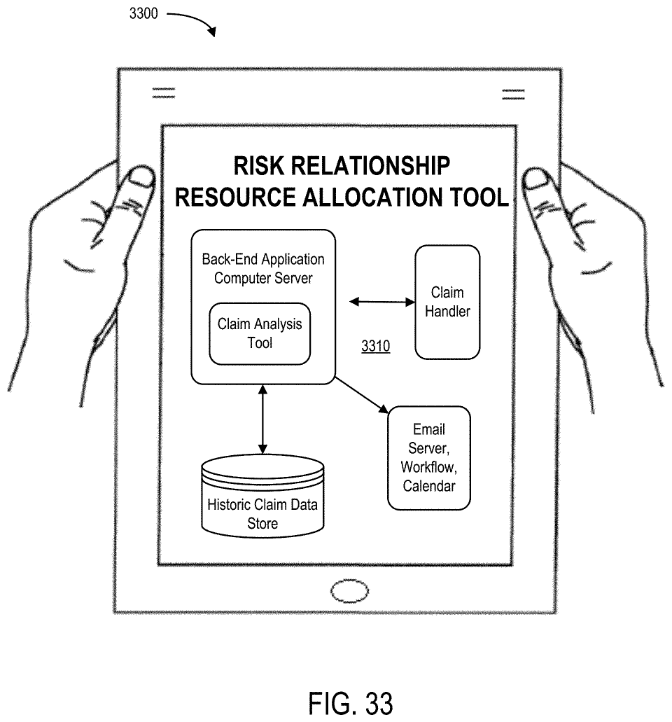

Further, the displays and devices illustrated herein are only provided as examples, and embodiments may be associated with any other types of user interfaces. For example, FIG. 33 illustrates a handheld tablet computer 3300 showing a risk relationship resource allocation tool display 3310 according to some embodiments. The resource allocation tool display 3310 might include user-selectable data that can be selected and/or modified by a user of the handheld computer 3300 to provide information about STD insurance claims.

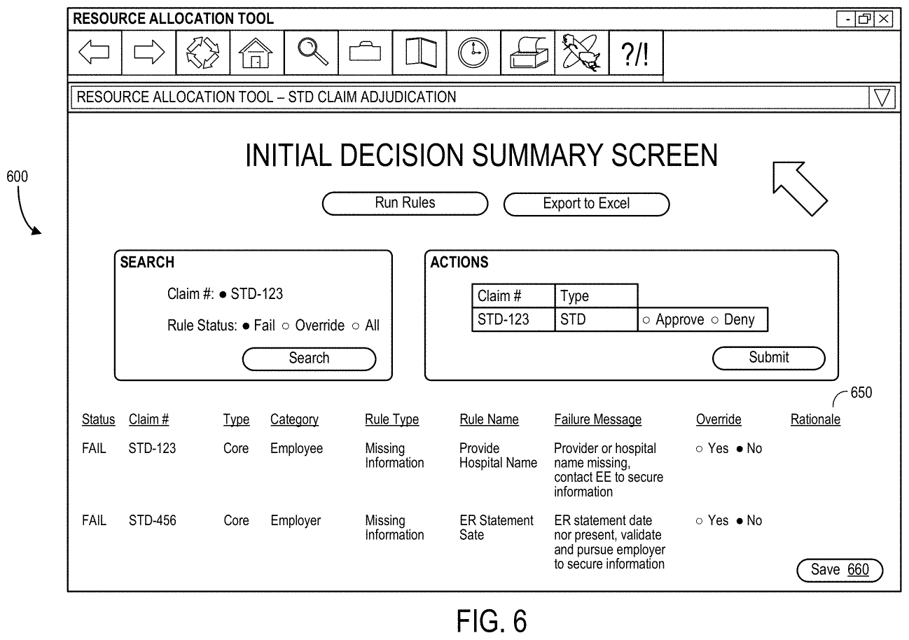

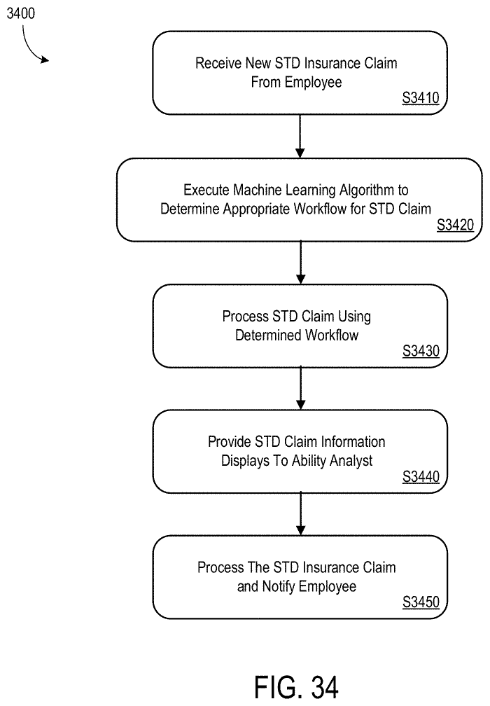

FIG. 34 illustrates an overall business process 3400 in accordance with some embodiments. At S3410, an insurer may receive a new STD insurance claim from a claimant (e.g., an employee of a customer of the insured). This information may be collected from various sources, including an employee, an employer, medical records, an incident report, etc. At S3420, a machine learning trained data science model is executed to determine an appropriate workflow for the STD claim (in accordance with any of the embodiments described herein). At S3430, the system may process the STD claim using the determined workflow. At S3440, claim information displays are provided to a claim handler (e.g., as described in connection with FIGS. 3 through 14). The insurer may then process the STD insurance claim as directed by the claim handler or ability analyst (e.g., by implementing a return-to-work strategy, etc.) and notify the employee.

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

What is claimed is:

1. A system to provide a risk relationship resource allocation tool via a back-end application computer server of an enterprise, comprising:
(a) a resource allocation data store containing electronic records that represent a plurality of requested resource allocations between the enterprise and a plurality of entities, wherein each electronic record includes an electronic record identifier and a set of resource allocation values associated with risk attributes that have been collected from the entities and service providers;
(b) the back-end application computer server, coupled to the resource allocation data store, programmed to:
automatically determine an initial duration for the selected requested resource allocation based on the shortest of: (i) an entity expected duration, (ii) a service provider expected duration, and (iii) a third-party guideline expected duration; and
(c) a communication port coupled to the back-end application computer server to facilitate a transmission of data with a remote device to provide a graphical interactive user interface display via a distributed communication network, the interactive user interface supporting the determined workflow.

2. The system of claim 1, wherein the enterprise is an insurer, the entities are employees of a client of the insurer, and the requested resource allocations are Short Term Disability (“STD”) insurance claims.

3. The system of claim 2, wherein the STD claim is associated with a first diagnostic code and a second diagnostic code, and the back-end application computer server recommends one of an expedited workflow and a non-expedited workflow for an ability analyst based at least in part on the first and second diagnostic codes.

4. The system of claim 3, wherein the recommendation is further based on at least one of: (i) an employer preference, (ii) a behavioral health indication, (iii) a claim reporting date, and (iv) a maternity claim indication.

5. A computerized method to provide a risk relationship resource allocation tool via a back-end application computer server of an enterprise, comprising:
receiving, by the back-end application computer server from a resource allocation data store, an indication of a selected requested resource allocation between the enterprise and an entity;
retrieving, by the back-end application computer server from a resource allocation data store, an electronic record associated with the selected requested resource allocation, including the set of resource allocation values associated with risk attributes, wherein the resource allocation data store contains electronic records that represent a plurality of requested resource allocations between the enterprise and a plurality of entities, and each electronic record includes an electronic record identifier and a set of resource allocation values associated with risk attributes that have been collected from the entities and service providers; and
automatically determining an initial duration for the selected requested resource allocation based on the shortest of: (i) an entity expected duration, (ii) a service provider expected duration, and (iii) a third-party guideline expected duration.

6. The method of claim 5, wherein the enterprise is an insurer, the entities are employees of a client of the insurer, the requested resource allocations are Short Term Disability (“STD”) insurance claims, the service provider is an attending physician, and the third-party guideline is an official disability guideline.

7. The method of claim 6, wherein the STD claim is associated with a first diagnostic code and a second diagnostic code, and the back-end application computer server recommends one of an expedited workflow and a non-expedited workflow for an ability analyst based at least in part on the first and second diagnostic codes.

8. The method of claim 7, wherein the recommendation is further based on at least one of: (i) an employer preference, (ii) a behavioral health indication, (iii) a claim reporting date, and (iv) a maternity claim indication.

9. A system to provide a risk relationship resource allocation tool via a back-end application computer server of an enterprise, comprising:
(a) a resource allocation data store containing electronic records that represent a plurality of requested resource allocations between the enterprise and a plurality of entities, wherein each electronic record includes an electronic record identifier and a set of resource allocation values associated with risk attributes that have been collected from the entities and service providers; and
(b) the back-end application computer server, coupled to the resource allocation data store, programmed to:
(i) receive an extension request for a requested resource allocation from an entity,
(ii) if it is determined that service provider records are not required for the requested resource allocation:
running a machine learning model to generate raw model output including duration likelihood scores and confidence thresholds for each of a range of durations,
if the highest likelihood score has a confidence threshold below a pre-determined value, generating a null model recommendation,
otherwise, using the shortest duration in the range of durations with the highest likelihood score as a model recommendation, and
combining the model recommendation with provider and guideline values to generate a final recommendation, and
(iii) if it is determined that service provider records are required for the requested resource allocation:
using the service provider records and a set of rules to automatically generate the final recommendation.

10. The system of claim 9, wherein the enterprise is an insurer, the entity is an employee of a client of the insurer, the requested resource allocations are Short Term Disability (“STD”) insurance claims, and the service provider records are medical records.

11. The system of claim 10, wherein the provider value is a physician return-to-work date and the guideline value is at least one of: (i) an official diagnostic guideline average value, and (ii) an official diagnostic guideline best practices value.

12. The system of claim 11, wherein the final recommendation is between the official diagnostic guideline average value and the official diagnostic guideline best practices value.

13. The system of claim 12, wherein a performance of the final recommendation is used in a feedback look to improve the machine learning model.

14. The system of claim 13, wherein the performance is associated with a manual review by an ability analyst to approve or deny the STD claim extension request.

15. The system of claim 14, wherein the set of rules include: (i) light occupational impairment rules, and (ii) medium and higher occupational impairment rules.

16. The system of claim 15, wherein the set of rules include at least one of: (i) closure rules, (ii) missing information rules, (iii) financial rules, (iv) exclusion rules, (v) medical rules, and (vi) occupational rules.

17. A non-transitory, computer-readable medium storing instructions, that, when executed by a processor, cause the processor to perform a method that provides a risk relationship resource allocation tool via a back-end application computer server of an enterprise, the method comprising:
receiving, at a back-end application computer server, an extension request for a requested resource allocation from an entity;
if it is determined that service provider records are not required for the requested resource allocation:
running a machine learning model to generate raw model output including duration likelihood scores and confidence thresholds for each of a range of durations,
if the highest likelihood score has a confidence threshold below a pre-determined value, generating a null model recommendation,
otherwise, using the shortest duration in the range of durations with the highest likelihood score as a model recommendation, and
combining the model recommendation with provider and guideline values to generate a final recommendation; and
if it is determined that service provider records are required for the requested resource allocation:
using the service provider records and a set of rules to automatically generate the final recommendation.

18. The medium of claim 17, wherein the enterprise is an insurer, the entity is an employee of a client of the insurer, the requested resource allocations are Short Term Disability (“STD”) insurance claims, and the service provider records are medical records.

19. The medium of claim 18, wherein the provider value is a physician return-to-work date and the guideline value is at least one of: (i) an official diagnostic guideline average value, and (ii) an official diagnostic guideline best practices value.

20. The medium of claim 19, wherein the final recommendation is between the official diagnostic guideline average value and the official diagnostic guideline best practices value.

21. The medium of claim 20, wherein a performance of the final recommendation is used in a feedback look to improve the machine learning model.

22. The medium of claim 21, wherein the performance is associated with a manual review by an ability analyst to approve or deny the STD claim extension request.
